(19) United States
(12) Patent Application Publication
(10) Pub. No.: US 2004/0259116 A1
(43) Pub. Date: Dec. 23, 2004

(54) Hairpin primer amplification

(75) Inventors: Kenneth B. Beckman, Alameda, CA (US); Ricardo Mancebo, Fremont, CA (US)

Correspondence Address: QUINE INTELLECTUAL PROPERTY LAW GROUP, P.C., P O BOX 458, ALAMEDA, CA, 94501, US

(73) Assignee: Gorilla Genomics, Inc., Alameda, CA

(21) Appl. No.: 10/766,313

(22) Filed: Jan. 27, 2004

Related U.S. Application Data
(60) Provisional application No. 60/443,626, filed on Jan. 28, 2003.
(60) Provisional application No. 60/458,246, filed on Mar. 26, 2003.

(51) Int. Cl. C12Q 1/68; C12P 19/34

(52) U.S. Cl. 435/6; 435/91.2

(57) ABSTRACT

Methods for amplifying nucleic acids (e.g., symmetric and asymmetric PCR methods) using a combination of linear and hairpin primers to enhance specificity are provided. The hairpin primer can be a fluorogenic hairpin primer, which facilitates detection of a nucleic acid target. Attendant compositions, systems, devices and kits are also provided.

### CROSS REFERENCE TO RELATED APPLICATIONS

This application is a non-provisional utility patent application claiming priority to and benefit of the following prior provisional patent applications: U.S. Ser. No. 60/443,626, filed Jan. 28, 2003, entitled “Hairpin Primer Amplification” by Beckman and Mancebo, and U.S. Ser. No. 60/458,246, filed Mar. 26, 2003, entitled “Hairpin Primer Amplification” by Beckman and Mancebo, each of which is incorporated herein by reference in its entirety for all purposes.

### FIELD OF THE INVENTION

This invention is in the field of nucleic acid amplification and detection, particularly asymmetric amplification and detection using fluorogenic hairpin primers.

### BACKGROUND OF THE INVENTION

The polymerase chain reaction (PCR) is a widely used technique in the life sciences, and in particular is becoming increasingly used in a real-time mode for the purposes of quantitation, so-called quantitive real-time PCR. The method typically involves the design of two primers, short oligonucleotides that are positioned relative to each other such that they are complementary to opposite strands of the intended amplicon (see FIG. 1A). Although powerful, the PCR process has always been plagued by problems of primer specificity, namely, the tendency of primers to prime and initiate synthesis at sites other than those intended, resulting in two related problems: 1) the non-specific amplification of unintended targets, and 2) the amplification of products formed from the direct interaction of two primers, so-called “primer dimers.” These unintended non-specific products limit the utility of quantitive real-time PCR for accurately quantifying the amount of the intended target product that is generated during amplification.

The present invention uses a combination of linear and hairpin primers to overcome the specificity issues noted above. A complete understanding of the invention will be obtained upon review of the following.

### SUMMARY OF THE INVENTION

The present invention provides methods in which a hairpin primer (e.g., a fluorogenic hairpin primer) is used in conjunction with a pair of linear primers for nucleic acid amplification (e.g., PCR amplification). The methods can be applied to various forms of PCR, including, but not limited to, real-time quantitative PCR, reverse transcription PCR, in situ PCR, and/or multiplex PCR, and can be used for single nucleotide discrimination (e.g., SNP detection, allele discrimination, and the like). Compositions, systems, and kits that relate to the methods are also features of the invention.

Thus, in a first general class of embodiments, the invention provides methods for amplifying a target nucleotide sequence or its reverse complement. In the methods, a template nucleic acid, a first linear primer, a second linear primer, and a hairpin primer are provided. The template nucleic acid comprises a first strand, the first strand comprising a target region that comprises the target nucleotide sequence or its reverse complement. The first linear primer comprises a region of identity to a 5′ subregion of the target region, while the second linear primer comprises a region of complementarity to a 3′ subregion of the target region. The 5′ subregion is located at the 5′ end of the target region and the 3′ subregion is located at the 3′ end of the target region; thus, the first and second linear primers define the two ends of the target region. The hairpin primer comprises a region of complementarity to a first subregion of the target region, which is 5′ of or at least partially overlapping the 3′ subregion. At least a portion of the target nucleotide sequence or its reverse complement is amplified by contacting the template nucleic acid, the first linear primer, the second linear primer and the hairpin primer and extending at least the hairpin primer.

In one class of embodiments, the hairpin primer comprises a 5′ arm, a loop and a 3′ arm. The 5′ arm and the 3′ arm are complementary to each other and are able to form a double-stranded duplex. At least a portion of the loop and the 3′ arm (preferably, the entire loop and 3′ arm) are complementary to the first subregion of the target region. Optionally, the hairpin primer also includes a fluorescent label and a quencher. The label emits a fluorescent signal. The label and the quencher are located within the hairpin primer such that the label emits a maximal fluorescent signal only when the 5′ and 3′ arms are not forming the double-stranded duplex.

The first and second linear primers can be provided at equal concentration, or an excess of the first linear primer can be provided. In one class of embodiments, the hairpin primer is fully nested; that is, the first subregion does not overlap the 3′ subregion. In these embodiments, the linear primers do not compete with the hairpin primer for annealing to the template. In another class of embodiments, the first subregion at least partially overlaps the 3′ subregion; the second linear primer and the hairpin primer thus compete for annealing to the first strand of the template.

The present invention also includes compositions, e.g., for practicing the methods herein or that are produced by the methods herein. For example, the invention provides a composition comprising a template nucleic acid, a first linear primer, a second linear primer, and a hairpin primer. The template nucleic acid comprises a first strand, the first strand comprising a target region that comprises a target nucleotide sequence or its reverse complement. The first linear primer comprises a region of identity to a 5′ subregion of the target region, while the second linear primer comprises a region of complementarity to a 3′ subregion of the target region. The 5′ subregion is located at the 5′ end of the target region and the 3′ subregion is located at the 3′ end of the target region; thus the first and second linear primers define the two ends of the target region. The hairpin primer comprises a region of complementarity to a first subregion of the target region, the first subregion being 5′ of or at least partially overlapping the 3′ subregion.

Kits form another aspect of the invention. Thus, one general class of embodiments provides a kit for use in amplifying a target nucleotide sequence or its reverse complement from a template nucleic acid strand that comprises a target region comprising the target nucleotide sequence or its reverse complement. The kit includes a first linear primer, a second linear primer, and a hairpin primer, packaged in one or more containers. The first linear primer comprises a region of identity to a 5′ subregion of the target region, while the second linear primer comprises a region of complementarity to a 3′ subregion of the target region. The hairpin primer comprises a region of complementarity to a first subregion of the target region, the first subregion being 5′ of or at least partially overlapping the 3′ subregion.

### BRIEF DESCRIPTION OF THE DRAWINGS

FIG. 1 schematically illustrates existing PCR primer compositions (Panels A-C) and some of the novel primer compositions of this invention (Panels D-E). Conventional PCR primers are schematically depicted in Panel A, hairpin PCR primers in Panel B, and fluorogenic hairpin PCR primers (FHPs) in Panel C. Nested fluorogenic hairpin PCR probe-primers are schematically depicted in Panel D and semi-nested fluorogenic hairpin PCR probe-primers in Panel E. Positions of the primers are schematically illustrated, with the hairpin primers in their folded conformations; this figure does not illustrate primer annealing or priming mechanism.

FIG. 2 Panels A-D schematically depict an asymmetric amplification with nested fluorogenic hairpin primer and linear primers.

FIG. 3 Panels A-D schematically depict an asymmetric amplification with semi-nested fluorogenic hairpin primer and linear primers.

### DEFINITIONS

Unless defined otherwise, all technical and scientific terms used herein have the same meaning as commonly understood by one of ordinary skill in the art to which the invention pertains. The following definitions supplement those in the art and are directed to the current application and are not to be imputed to any related or unrelated case, e.g., to any commonly owned patent or application. Although any methods and materials similar or equivalent to those described herein can be used in the practice for testing of the present invention, the preferred materials and methods are described herein. Accordingly, the terminology used herein is for the purpose of describing particular embodiments only, and is not intended to be limiting.

A “label” is a moiety that facilitates detection of a molecule. Common labels in the context of the present invention include fluorescent and colorimetric labels.

A “quencher” is a moiety that alters a property of a label (typically, a fluorescent label) when it is in proximity to the label. The quencher can actually quench an emission, but it does not have to, i.e., it can simply alter some detectable property of the label, or, when proximal to the label, cause a different detectable property than when not proximal to the label. A quencher can be e.g., an acceptor fluorophore that operates via energy transfer and re-emits the transferred energy as light; other similar quenchers do not re-emit transferred energy as light.

A “molecular beacon” (MB) is an oligonucleotide or PNA which, under appropriate hybridization conditions (e.g., free in solution), self-hybridizes to form a stem and loop structure. The MB has a label and a quencher at the termini of the oligonucleotide or PNA; thus, under conditions that permit intra-molecular hybridization, the label is typically quenched (or otherwise altered) by the quencher. Under conditions where the MB does not display intra-molecular hybridization (e.g., when bound to a target nucleic acid), the MB label is unquenched.

The term “nucleic acid” encompasses any physical string of monomer units that can be corresponded to a string of nucleotides, including a polymer of nucleotides (e.g., a typical DNA or RNA polymer), PNAs, modified oligonucleotides (e.g., oligonucleotides comprising bases that are not typical to biological RNA or DNA in solution, such as 2′-O-methylated oligonucleotides), and the like. A nucleic acid can be e.g., single-stranded or double-stranded.

A “nucleotide sequence” is a polymer of nucleotides (an oligonucleotide, a DNA, a nucleic acid, etc.) or a character string representing a nucleotide polymer, depending on context. Either the given nucleic acid or the complementary nucleic acid can be determined from any specified nucleotide sequence.

An “oligonucleotide” is a polymer comprising two or more nucleotides. The polymer can additionally comprise non-nucleotide elements such as labels, quenchers, blocking groups, or the like. The nucleotides of the oligonucleotide can be natural or non-natural and can be unsubstituted, unmodified, substituted or modified. The nucleotides can be linked by phosphodiester bonds, or by phosphorothioate linkages, methylphosphonate linkages, boranophosphate linkages, or the like.

A “peptide nucleic acid” (PNA) is a polymer comprising two or more peptide nucleic acid monomers. The polymer can additionally comprise elements such as labels, quenchers, blocking groups, or the like. The monomers of the PNA can be unsubstituted, unmodified, substituted or modified.

A “primer” is a nucleic acid that contains a sequence complementary to a region of a template nucleic acid strand and that primes the synthesis of a strand complementary to the template (or a portion thereof). Primers are typically, but need not be, relatively short, chemically synthesized oligonucleotides (typically, deoxyribonucleotides). In an amplification, e.g., a PCR amplification, a pair of primers typically define the 5′ ends of the two complementary strands of the nucleic acid target that is amplified. In order to be extendable by a standard polymerase, a primer typically has a free 3′ hydroxyl group.

A “hairpin primer” is a single nucleic acid molecule that comprises self-complementary sequences and is thus capable of folding back on itself under appropriate conditions. Typically, a hairpin primer comprises a pair of complementary sequences that are able to base pair and form a double-stranded stem, where the complementary sequences are connected by one or more nucleotides, PNA monomers, or the like that are able to form a loop or sharp bend (or otherwise allow base pairing to occur between the complementary sequences forming the stem). Like any primer, a hairpin primer typically has a free 3′ hydroxyl.

A “linear primer” is a single-stranded nucleic acid molecule that does not comprise self-complementary sequences and thus does not fold back on itself to form a defined secondary structure.

A “target region” is a region of a nucleic acid that is to be amplified, detected or both.

The “Tm” (melting temperature) of a nucleic acid duplex under specified conditions is the temperature at which half of the base pairs are disassociated and half are associated.

“5′ to 3′ nuclease activity” is an enzymatic activity that includes either a 5′ to 3′ exonuclease activity, whereby nucleotides are removed from the 5′ end of a nucleic acid strand (e.g., an oligonucleotide) in a sequential manner; or a 5′ to 3′ endonuclease activity, wherein cleavage occurs more than one nucleotide from the 5′ end; or both. An example of 5′ to 3′ endonuclease activity is the flap endonuclease activity exhibited by the Thermus aquaticus DNA polymerase Taq.

The 5′ to 3′ nuclease activity of a polymerase “substantially lacking 5′ to 3′ nuclease activity” or which is “nuclease-free” is about 20% or less (e.g., 10% or less or 5% or less) than that of the Taq DNA polymerase from Thermus aquaticus under typical reaction conditions (e.g., typical primer extension conditions for the polymerase). Optionally, the nuclease activity of the nuclease-free enzyme can be completely absent, i.e., undetectable under such typical reaction conditions. Thermus aquaticus Taq is described, e.g., in U.S. Pat. No. 4,889,818 and U.S. Pat. No. 5,079,352. Example DNA polymerases substantially lacking 5′ to 3′ nuclease activity include, e.g., any DNA polymerase having undetectable 5′ to 3′ nuclease activity under typical primer extension conditions for that polymerase; the Klenow fragment of E. coli DNA polymerase I; a Thermus aquaticus Taq lacking the N-terminal 235 amino acids (e.g., as described in U.S. Pat. No. 5,616,494); and/or a thermostable DNA polymerase having sufficient deletions (e.g., N-terminal deletions), mutations, or modifications so as to eliminate or inactivate the domain responsible for 5′ to 3′ nuclease activity.

A primer that is “resistant to 5′ to 3′ nuclease activity” is cleaved more slowly under typical reaction conditions for a given 5′ to 3′ nuclease than is a corresponding primer comprising only the four conventional deoxyribonucleotides (A, T, G, and/or C) and phosphodiester linkages.

A variety of additional terms are defined or otherwise characterized herein.

### DETAILED DESCRIPTION

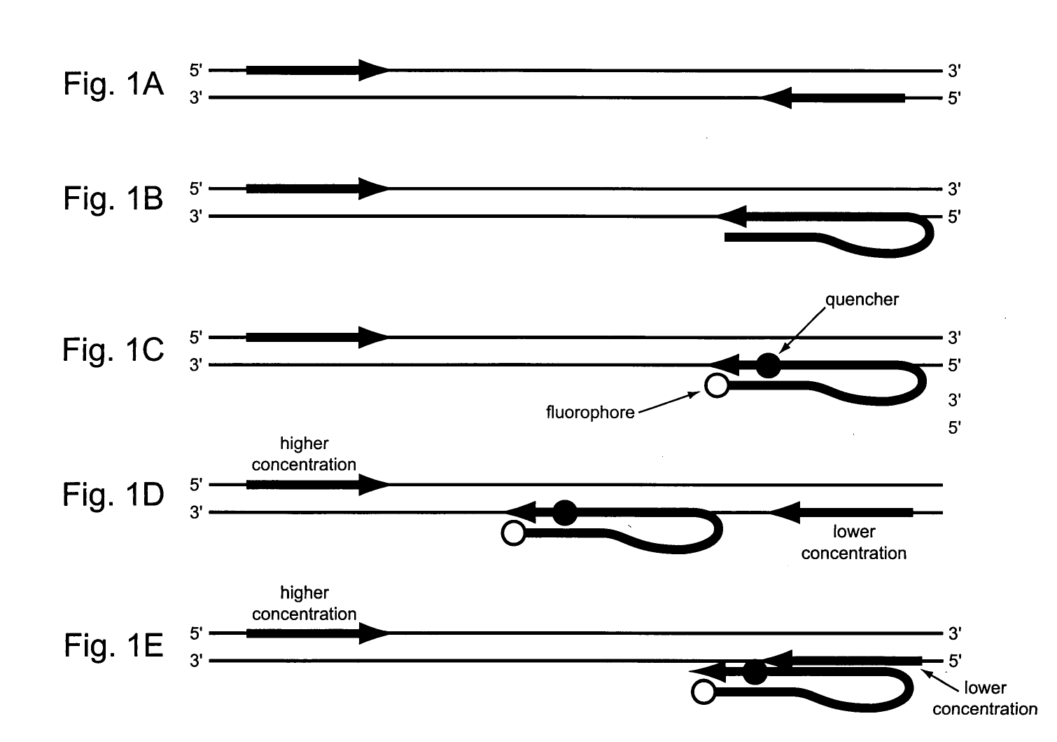

Hairpin primers for use in polymerase chain reaction (PCR) amplification have been described in the art (see, e.g., Kaboev et al. (2000) Nuc. Acids Res. 28 (21):e94), and claims for their utility in decreasing non-specific amplification reaction products have been made. Such hairpin primers possess a “stem and loop” structure, wherein the 5′ and 3′ ends (or “arms”) of the oligonucleotide are self-complementary, causing the primer to hybridize to itself and form a hairpin structure (FIG. 1B). Typically, self-complementary 5′ and 3′ arms are 5-9 nucleotides in length (but the arms can be shorter or longer, depending on the desired application). In this regard, these structures are similar to molecular beacons. (For details regarding methods of making and using molecular beacons, see, e.g., Leone et al. (1995) “Molecular beacon probes combined with amplification by NASBA enable homogenous real-time detection of RNA.” Nucleic Acids Res. 26:2150-2155; Tyagi and Kramer (1996) “Molecular beacons: probes that fluoresce upon hybridization” Nature Biotechnology14:303-308; Blok and Kramer (1997) “Amplifiable hybridization probes containing a molecular switch” Mol Cell Probes 11:187-194; Hsuih et al. (1997) “Novel, ligation-dependent PCR assay for detection of hepatitis C in serum” J Clin Microbiol 34:501-507; Kostrikis et al. (1998) “Molecular beacons: spectral genotyping of human alleles” Science 279:1228-1229; Sokol et al. (1998) “Real time detection of DNA:RNA hybridization in living cells” Proc. Natl. Acad. Sci. U.S.A. 95:11538-11543; Tyagi et al. (1998) “Multicolor molecular beacons for allele discrimination” Nature Biotechnology 16:49-53; Bonnet et al. (1999) “Thermodynamic basis of the chemical specificity of structured DNA probes” Proc. Natl. Acad. Sci. U.S.A. 96:6171-6176; Fang et al. (1999) “Designing a novel molecular beacon for surface-immobilized DNA hybridization studies” J. Am. Chem. Soc. 121:2921-2922; Marras et al. (1999) “Multiplex detection of single-nucleotide variation using molecular beacons” Genet. Anal. Biomol. Eng. 14:151-156; Vet et al. (1999) “Multiplex detection of four pathogenic retroviruses using molecular beacons” Proc. Natl. Acad. Sci. U.S.A. 96:6394-6399; patent application USSN PCT/US01/13719; U.S. Pat. No. 5,925,517 (Jul. 20, 1999) to Tyagi et al. entitled “Detectably labeled dual conformation oligonucleotide probes, assays and kits;” U.S. Pat. No. 6,150,097 to Tyagi et al (Nov. 21, 2000) entitled “Nucleic acid detection probes having non-FRET fluorescence quenching and kits and assays including such probes” and U.S. Pat. No. 6,037,130 to Tyagi et al (Mar. 14, 2000), entitled “Wavelength-shifting probes and primers and their use in assays and kits.”) However, unlike molecular beacons, hairpin primers are extendable at their 3′ ends.

In working with these hairpin primers, we have discovered that although they can indeed increase specificity in amplification reactions, they are frequently poor primers as a result, amplifying with an efficiency that is far from the ideal goal of a doubling in amplicon with each cycle of PCR. This decreased efficiency is particularly apparent when attempting to amplify from low copies of target molecule, as is frequently the case in quantitative PCR applications.

In one embodiment taught in the art, hairpin primers have been designed with a fluorophore on one arm of the stem, and a quencher moiety on the other arm, with the result that as the primer is incoporated into the amplicon, fluorescent signal is generated (so-called “fluorogenic hairpin primers”, or FHPs, see FIG. 1C). See, e.g., U.S. Pat. No. 6,277,607 (Aug. 21, 2001) to Tyagi et al. entitled “High specificity primers, amplification methods and kits”; U.S. Pat. No. 5,866,336 (Feb. 2, 1999) to Nazarenko et al. entitled “Nucleic acid amplification oligonucleotides with molecular energy transfer labels and methods based thereon”; and Nazarenko et al. (1997) “A closed tube format for amplification and detection of DNA based on energy transfer” Nucl. Acids Res. 25:2516-2521. Use of such a fluorogenic hairpin primer can provide a convenient means of detecting an amplicon including the FHP, but FHPs suffer from the same decreased efficiency of amplification as non-fluorogenic hairpin primers.

We have discovered a novel way to overcome the inefficiency associated with hairpin primers (including FHPs), by designing a set of three primers, composed of two linear primers and a FHP, in which the FHP is located internally to the two linear primers, in a nested or semi-nested arrangement (see FIGS. 1D and 1E). As a result, we have found that the combination of three primers amplifies efficiently, under conditions in which the FHP alone may not always amplify efficiently, indicating that the FHP does not always take part in the early cycles of a given amplification reaction. In other words, the internal FHP acts partly as a beacon-like probe (in that it specifically detects the amplicon) and partly as a primer (in that it is extendable and is incorporated into an amplicon itself, thereby generating fluorescence).

Furthermore, we describe concentrations of the two linear outside primers (the first and second linear primers) that enhance the generation of fluorescent signal. In short, by employing an asymmetric ratio of the two outside linear primers, such that the concentration of the outside linear primer that is complementary to the same strand as the FHP (the second linear primer) (e.g., 150 nM) is lower than that of the outside linear primer that is complementary to the opposite strand as the FHP (the first linear primer) (e.g., 600 nM), the incorporation of the FHP (provided, e.g., at 100 nM) into product is enhanced, thereby increasing the generation of fluorescence. Furthermore, we have found that the sequences of the FHP and the outside linear primer that is complementary to the same strand as the FHP (the second linear primer) can have significant sequence identity, such that they both hybridize to the same sequences, with the 3′ terminus of the FHP typically (but not necessarily) positioned 3′ to the 3′ terminus of the second linear primer (FIG. 1E). A benefit of this particular embodiment (as opposed to the fully nested embodiment in which the outside linear primers and the FHP do not overlap, FIG. 1D) is that unintended cleavage of the FHP by a polymerase extending the linear primer lying upstream of it is much less likely when the FHP and linear primer are competitive in their hybridization to target or amplicon molecules. As illustrated in FIG. 1E, a staggered positioning of the hairpin primer and the outside linear primer, e.g., with the 3′ ends of the hairpin primer 3′ to the linear primer on the same strand (the second linear primer), results in competition between these two primers and prevents co-hybridization.

The advantages of this invention are as follows. First, the use of a highly specific hairpin primer is enabled, because the inefficient priming that is often seen with such stable hairpin primer structures, and which thereby limits their utility, is overcome through the use of a second outside linear primer 5′ to the FHP on the same strand, which delivers effective amplification efficiency. At the same time, primer dimers formed by these linear outside primers do not contain sequences that are complementary to the nested 3′ end of the internal FHP, thereby reducing the likelihood that the FHP will detect primer-dimers generated from linear primers. Second, by nesting the 3′ end of a hairpin primer internal to the 3′ end of the second linear primer, the specificity of the reaction is enhanced, because the internal FHP will only amplify a primary amplicon sequence.

Third, it is possible to use a low concentration of the FHP, since it is only required to generate signal in a secondary internal amplification, and not to provide efficient amplification of the primary target molecule, and can therefore be used at concentrations which would otherwise result in poor amplification efficiency. This can result in cost savings and increased specificity.

Fourth, by positioning the FHP on the same strand, 3′ downstream, and overlapping by only a few nucleotides one of the two outside linear primers, one ensures that cleavage of this FHP by polymerase will not occur, thereby limiting undesirable “Taqman”-like activity.

The advantages and disadvantages of existing primer compositions and some example compositions of this invention can be summarized as follows. Conventional PCR primers (FIG. 1A) provide efficient amplification but suffer from non-specificity and primer-dimer formation and are non-fluorogenic. Hairpin PCR primers (FIG. 1B) provide improved specificity and decreased primer-dimer formation, but suffer from inefficient amplification and are non-fluorogenic. Fluorogenic hairpin primers (FIG. 1C) provide improved specificity and decreased primer-dimer formation and are fluorogenic, but suffer from inefficient amplification. The nested fluorogenic hairpin PCR probe-primers of this invention (FIG. 1D) provide efficient amplification, improved specificity, and decreased primer-dimer formation and are fluorogenic; but have some potential for probe-primer cleavage. The semi-nested fluorogenic hairpin PCR probe-primers of this invention (FIG. 1E) provide efficient amplification, improved specificity and decreased primer-dimer formation, are fluorogenic, and offer little or no potential for probe-primer cleavage.

Amplification with Hairpin Primers

One aspect of the present invention provides new amplification strategies (e.g., symmetric and asymmetric PCR strategies) using a combination of linear and hairpin primers to enhance specificity. In some embodiments, the hairpin primer is fluorogenic, facilitating combined amplification and detection of a nucleic acid target.

One general class of embodiments provides methods for amplifying a target nucleotide sequence or its reverse complement. In the methods, a template nucleic acid, a first linear primer, a second linear primer, and a hairpin primer are provided. The template nucleic acid comprises a first strand, the first strand comprising a target region that comprises the target nucleotide sequence or its reverse complement. The first linear primer comprises a region of identity to a 5′ subregion of the target region, while the second linear primer comprises a region of complementarity to a 3′ subregion of the target region. The hairpin primer comprises a region of complementarity to a first subregion of the target region, which is 5′ of or at least partially overlapping the 3′ subregion. At least a portion of the target nucleotide sequence or its reverse complement is amplified by contacting the template nucleic acid, the first linear primer, the second linear primer and the hairpin primer and extending at least the hairpin primer. (Typically, the first and/or second linear primer is also extended.) The 5′ subregion is located at the 5′ end of the target region and the 3′ subregion is located at the 3′ end of the target region; thus, the first and second linear primers define the two ends of the target region. The hairpin primer can be extended by any of a variety of techniques known in the art, e.g., by PCR. Typically, a polymerase catalyzes template-dependent extension of the primer(s), in the presence of deoxyribonucleoside triphosphates, an aqueous buffer, appropriate salts and metal cations, and the like, to form one or more double-stranded extension products.

In one class of embodiments, the hairpin primer comprises a 5′ arm, a loop and a 3′ arm. The 5′ arm and the 3′ arm are complementary to each other and are able to form a double-stranded duplex. At least a portion of the loop and the 3′ arm are complementary to the first subregion of the target region. The entire 3′ arm is typically complementary to the first subregion; the entire loop is typically but not necessarily also complementary to the first subregion. The 5′ arm can be partially or entirely complementary to the target region as well, but typically is not. Thus, in a preferred class of embodiments, the entire loop and 3′ arm are complementary to the first subregion.

In one class of embodiments, the hairpin primer consists of the 5′ arm, loop and 3′ arm, with at least a portion of the loop and the 3′ arm being complementary to the first subregion; that is, the double-stranded duplex formed by the arms is blunt-ended with no overhanging 3′ or 5′ nucleotides. In other embodiments, additional sequence complementary to the target follows the 3′ arm; this 3′ overhanging sequence may encourage hybridization to the target region, but may also decrease specificity and/or encourage primer-dimer formation.

One class of embodiments provides fluorogenic hairpin primers, in which the hairpin primer also includes a fluorescent label and a quencher. The label emits a fluorescent signal. The label and the quencher are located within the hairpin primer such that the label emits a maximal fluorescent signal only when the 5′ and 3′ arms are not forming the double-stranded duplex. For example, the label can emit the maximal signal when the loop and 3′ arm are annealed to the first subregion of the target region. Thus, annealing of the hairpin primer to the first strand of the template prevents formation of the double-stranded duplex and permits emission (and thus detection) of the maximal fluorescent signal. Extension of the hairpin primer to form an extended primer (e.g., by polymerase beginning in the annealing step of the PCR cycle and continuing in the extension step) adds more nucleotides to the primer that are complementary to the template strand and thus stabilizes binding of the extended hairpin primer to the first strand. Thus, incorporation of the hairpin primer into a double-stranded product of the extending step prevents formation of the double-stranded duplex and permits emission of the maximal fluorescent signal. Similarly, annealing of an extended hairpin primer produced by the extending step to a complementary nucleic acid strand prevents formation of the double-stranded duplex and thus permits emission of the maximal fluorescent signal.

In one class of embodiments, the fluorescent signal emitted by the label is detected. The signal can be detected at any suitable point or points during the amplification; for example, the signal can be detected during each annealing step during PCR cycles. As another example, the signal can be detected after each extension step during PCR, and/or at any other point during the PCR cycles except during the denaturation step. In some embodiments, the intensity of the fluorescent signal is measured (e.g., at each PCR cycle for quantitative real-time PCR).

In another class of embodiments, the method facilitates amplification and detection of two or more nucleic acid targets simultaneously (e.g., by multiplex PCR). In this class of embodiments, a first linear primer, a second linear primer, a hairpin primer, and a template nucleic acid are provided for each of two or more different target regions, and each hairpin primer is extended (typically, the first and/or second linear primers are also extended). The signals from the different FHPs are typically distinguishable from each other, such that information about each different target can be acquired (e.g., the label on each FHP can fluoresce at a different wavelength). Thus, in one embodiment, each hairpin primer comprises a different fluorescent label that emits a fluorescent signal distinguishable from that of each of the other fluorescent labels, and the fluorescent signal from the label on each of the hairpin primers is detected.

The first and second linear primers can be provided at equal concentration. As noted above, however, providing an excess of the first linear primer can enhance incorporation of the hairpin primer (and thus increase sensitivity of target detection by a FHP). Thus, in one class of embodiments, the first linear primer is provided a concentration that is at least about 1.3 times (e.g., at least about two times, at least about three times, at least about four times, at least about five times, at least about six times, or more) the concentration of the second linear primer. The concentration of the hairpin primer is typically, but not necessarily, equal to or less than that of the second linear primer. Optimal concentrations of each primer can readily be determined by one of skill, e.g., by varying the concentrations at which the primers are provided and then selecting a concentration of each primer that maximizes specific fluorescent signal from the FHP and that in general improves PCR performance (e.g., maximizes production of the desired product(s) and/or optimizes efficiency of amplification).

In one class of embodiments, a polymerase substantially lacking 5′ to 3′ nuclease activity is used to extend at least the hairpin primer. A polymerase substantially lacking 5′ to 3′ nuclease activity has a 5′ to 3′ nuclease activity that is about twenty percent or less than that of the Thermus aquaticus Taq DNA polymerase under typical reaction conditions (e.g., typical primer extension conditions for the polymerase, e.g., typical PCR conditions). In other words, the 5′ to 3′ nuclease activity of the polymerase is about one-fifth, or less than about one-fifth, the 5′ to 3′ nuclease activity of Taq. For example, the polymerase can have a 5′ to 3′ nuclease activity that is ten percent or less (e.g., five percent or less) than that of Taq under typical reaction conditions. Optionally, the polymerase has no detectable 5′ to 3′ nuclease activity under typical reaction conditions (e.g., typical PCR conditions). In other embodiments, the polymerase has 5′ to 3′ nuclease activity (e.g., Taq polymerase).

In one class of embodiments, the hairpin primer is resistant to 5′ to 3′ nuclease activity. A variety of nuclease-resistant hairpin primers can be created, e.g., comprising modified nucleotides or modified intemucleotide linkages such as those used in the synthesis of antisense oligonucleotides. For example, the hairpin primer can comprise a peptide nucleic acid (PNA). As another example, the hairpin primer can comprise one or more 2′-O-methyl nucleotides (e.g., a hairpin primer comprising standard deoxyribonucleotides can also comprise one or more 2′-O-methyl nucleotides, e.g., at its 5′ end, or a hairpin primer can consist entirely of 2′-O-methyl nucleotides). The hairpin primer can contain one or more modified internucleotide linkages, as well as or instead of one or more modified nucleotides. For example, the hairpin primer can comprise one or more phosphorothioate linkages (oligonucleotides comprising such linkages are sometimes called S-oligos). A hairpin primer can comprise, e.g., only phosphorothioate linkages or a mixture of phosphodiester and phosphorothioate linkages. As other examples, the hairpin primer can comprise one or more methylphosphonate linkages, one or more boranophosphate linkages, or the like. Combinations of typical nuclease resistance modification strategies can also be employed; for example, a nuclease resistant hairpin primer can comprise both 2′-O-methyl nucleotides and phosphorothioate linkages. In other embodiments, the hairpin primer includes only standard nucleotides and phosphodiester bonds and is not resistant to 5′ to 3′ nuclease activity.

In one class of embodiments, the primers are fully nested; that is, the first subregion does not overlap the 3′ subregion. In these embodiments, the primers do not compete for annealing to the template. Optionally, unintended cleavage of the hairpin primer by a polymerase extending a second linear primer annealed 5′ of the hairpin primer can be minimized by using a polymerase substantially lacking 5′ to 3′ nuclease activity and/or by using a hairpin primer that is resistant to 5′ to 3′ nuclease activity, as described above.

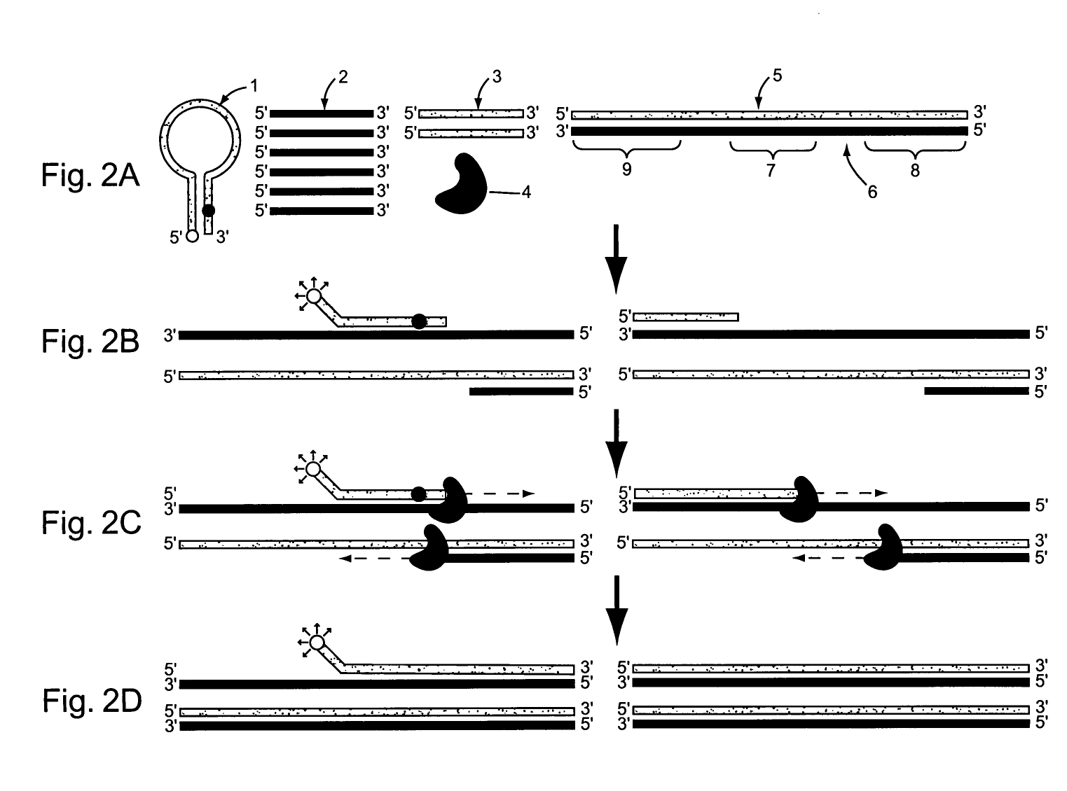

An example of an amplification (e.g., a PCR amplification) using an asymmetric ratio of first and second linear primers and a FHP, where the 3′ and first subregions do not overlap, is schematically illustrated in FIG. 2. Panel A depicts FHP 1 (optionally, a nuclease resistant FHP) in its hairpin conformation, in which the fluorescent label (open circle) is quenched by the quencher (filled circle); first linear primer 2, which is present in excess (e.g., at least threefold excess as depicted) of second linear primer 3; polymerase 4 (optionally, a polymerase substantially lacking 5′ to 3′ nuclease activity); first strand 6 of the template nucleic acid; and complementary strand 5 of the template. First strand 6 includes 5′ subregion 8, 3′ subregion 9, and first subregion 7, which do not overlap. As depicted, the template nucleic acid includes only the target region and is identical to one of the double-stranded extension products, but as will be evident to one of skill in the art, the template initially provided can be, e.g., single-stranded or double-stranded and can contain additional sequences 5′ and/or 3′ of the target region that are not amplified. As illustrated, the loop and 3′ arms of the FHP are complementary to first subregion 7 of first strand 6 of the template, first linear primer 2 is identical to 5′ subregion 8 of first strand 6, and second linear primer 3 is complementary to 3′ subregion 9 of first strand 6. The double-stranded template (or double-stranded extension product of a previous cycle) is denatured, e.g., at temperatures greater than about 90° C. The temperature is decreased (e.g., to 50-75° C.), and one or more primers anneal to their respective strand of the template. As depicted in Panel B, when the loop and 3′ arm of the FHP are bound to the complementary first subregion of the target, the fluorescent label and quencher are separated, resulting in a maximal signal from the label. Panel C depicts extension of the FHP and the first and second linear primers, while Panel D depicts extension products, which can be used as template in another cycle if desired.

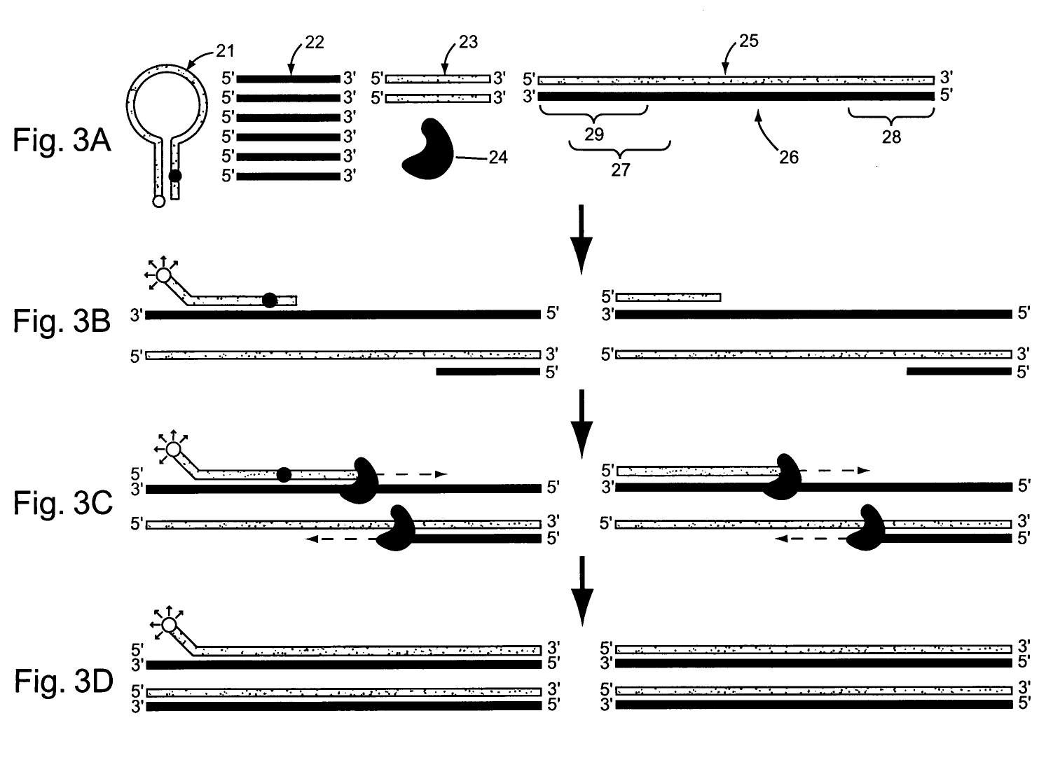

In another class of embodiments, the first subregion at least partially overlaps the 3′ subregion. The second linear primer and the hairpin primer thus compete for annealing to the first strand of the template. The first and 3′ subregions can partially or completely overlap, although typically the 5′ end of the first subregion is 5′ of the 5′ end of the 3′ subregion and the 3′ end of the first subregion is 5′ of the 3′ end of the second subregion (see, e.g., FIG. 3A). Preferably, the length of the overlap between the first subregion and the 3′ subregion is chosen such that the hairpin primer is unlikely to anneal to a primer dimer generated between the first and second linear primers (e.g., preferably the overlap consists of a number of nucleotides of such nucleotide sequence composition that the Tm of the hairpin primer-putative primer dimer strand is less than about 50° C. for a PCR annealing temperature of 55° C.).

An example of an amplification (e.g., a PCR amplification) using an asymmetric ratio of first and second linear primers and a FHP, where the 3′ and first subregions partially overlap, is schematically illustrated in FIG. 3. Panel A depicts FHP 21 (optionally, a nuclease resistant FHP) in its hairpin conformation, in which the fluorescent label (open circle) is quenched by the quencher (filled circle); first linear primer 22, which is present in excess (e.g., at least threefold excess as depicted) of second linear primer 23; polymerase 24 (optionally, a polymerase substantially lacking 5′ to 3′ nuclease activity); first strand 26 of the template nucleic acid; and complementary strand 25 of the template. First strand 26 includes 5′ subregion 28, 3′ subregion 29, and first subregion 27, which partially overlaps 3′ subregion 29. As depicted, the template nucleic acid includes only the target region and is identical to one of the double-stranded extension products, but as will be evident to one of skill in the art, the template initially provided can be, e.g., single-stranded or double-stranded and can contain additional sequences 5′ and/or 3′ of the target region that are not amplified. As illustrated, the loop and 3′ arms of the FHP are complementary to first subregion 27 of first strand 26 of the template, first linear primer 22 is identical to 5′ subregion 28 of first strand 26, and second linear primer 23 is complementary to 3′ subregion 29 of first strand 26. The double-stranded template (or double-stranded extension product of a previous cycle) is denatured, e.g., at temperatures greater than about 90° C. The temperature is decreased (e.g., to 50-75° C.), and one or more primers anneal to their respective strand of the template. As depicted in Panel B, when the loop and 3′ arm of the FHP are bound to the complementary first subregion of the target, the fluorescent label and quencher are separated, resulting in a maximal signal from the label. Panel C depicts extension of the FHP and the first and second linear primers, while Panel D depicts extension products, which can be used as template in another cycle if desired.

The template nucleic acid can be, e.g., any single-stranded or double-stranded DNA. For example, in one embodiment, the template nucleic acid is a single-stranded DNA product of a reverse transcription reaction (e.g., FHPs can be conveniently used to detect RNA targets by reverse transcription-PCR, including quantitative reverse transcription-PCR). As other examples, the template nucleic acid can be a synthetic oligonucleotide, a double-stranded cDNA, a single-stranded PCR product, or a double-stranded PCR product or can comprise genomic DNA.

The template nucleic acid can be derived from essentially any source, including, but not limited to: a human; an animal; a plant; a bacterium; a virus; cultured cells or culture medium; a tissue or fluid, e.g., from a patient, such as skin, blood, sputum, urine, stool, semen, or spinal fluid; a tumor; a biopsy; and/or the like

Compositions, Systems and Kits

The present invention also includes compositions, systems, and kits, e.g., for practicing the methods herein or which are produced by the methods herein.

In one general class of embodiments, the invention provides a composition comprising a template nucleic acid, a first linear primer, a second linear primer, and a hairpin primer. The template nucleic acid comprises a first strand, the first strand comprising a target region that comprises a target nucleotide sequence or its reverse complement. The first linear primer comprises a region of identity to a 5′ subregion of the target region, while the second linear primer comprises a region of complementarity to a 3′ subregion of the target region. The 5′ subregion is located at the 5′ end of the target region and the 3′ subregion is located at the 3′ end of the target region; thus the first and second linear primers define the two ends of the target region. The hairpin primer comprises a region of complementarity to a first subregion of the target region, the first subregion being 5′ of or at least partially overlapping the 3′ subregion.

In one class of embodiments, the hairpin primer comprises a 5′ arm, a loop and a 3′ arm. The 5′ arm and the 3′ arm are complementary to each other and are able to form a double-stranded duplex. At least a portion of the loop and the 3′ arm are complementary to the first subregion of the target region. The entire 3′ arm is typically complementary to the first subregion; the entire loop is typically but not necessarily also complementary to the first subregion. The 5′ arm can be partially or entirely complementary to the target region as well, but typically is not. Thus, in a preferred class of embodiments, the entire loop and 3′ arm are complementary to the first subregion.

In one class of embodiments, the hairpin primer consists of the 5′ arm, loop and 3′ arm, with at least a portion of the loop and the 3′ arm being complementary to the first subregion; that is, the double-stranded duplex formed by the arms is blunt-ended with no overhanging 3′ or 5′ nucleotides. In other embodiments, additional sequence complementary to the target follows the 3′ arm; this 3′ overhanging sequence may encourage hybridization to the target region, but may also decrease specificity and/or encourage primer-dimer formation.

In one class of embodiments, the hairpin primers are fluorogenic hairpin primers. In this class of embodiments, the hairpin primer also includes a fluorescent label and a quencher. The label emits a fluorescent signal. The label and the quencher are located within the hairpin primer such that the label emits a maximal fluorescent signal only when the 5′ and 3′ arms are not forming the double-stranded duplex. For example, the label can emit the maximal signal when the loop and 3′ arm are annealed to the first subregion of the target region. Thus, in one embodiment, the hairpin primer has been extended to form a double-stranded product. This incorporation of the hairpin primer into the product prevents formation of the double-stranded duplex and thus permits emission of the maximal fluorescent signal. Similarly, in another embodiment, the hairpin primer has been extended to form an extended hairpin primer, and the extended hairpin primer is annealed to a complementary nucleic acid strand. This prevents formation of the double-stranded duplex and thus permits emission of the maximal fluorescent signal. The composition optionally also includes an extended first linear primer, an extended second linear primer, a double-stranded product formed by extension of the first or second linear primer, or the like.

The first and second linear primers can be provided at equal concentration. As noted above, however, providing an excess of the first linear primer can enhance incorporation of the hairpin primer (and thus increase sensitivity of target detection by a FHP). Thus, in one class of embodiments, the first linear primer is provided a concentration that is at least about 1.3 times (e.g., at least about two times, at least about three times, at least about four times, at least about five times, at least about six times, or more) the concentration of the second linear primer. The concentration of the hairpin primer is typically, but not necessarily, equal to or less than that of the second linear primer.

In one class of embodiments, the primers are fully nested; that is, the first subregion does not overlap the 3′ subregion. In these embodiments, the primers do not compete for annealing to the template. In another class of embodiments, the first subregion at least partially overlaps the 3′ subregion. The second linear primer and the hairpin primer thus compete for annealing to the first strand of the template. The first and 3′ subregions can partially or completely overlap, although typically the 5′ end of the first subregion is 5′ of the 5′ end of the 3′ subregion and the 3′ end of the first subregion is 5′ of the 3′ end of the 3′ subregion (see, e.g., FIG. 3A).

The composition can optionally include a polymerase. In some embodiments, the polymerase has 5′ to 3′ nuclease activity (e.g., Taq polymerase). In other embodiments, the polymerase substantially lacks 5′ to 3′ nuclease activity. A polymerase substantially lacking 5′ to 3′ nuclease activity has a 5′ to 3′ nuclease activity that is about twenty percent or less than that of the Thermus aquaticus Taq DNA polymerase under typical reaction conditions (e.g., typical primer extension conditions for the polymerase, e.g., typical PCR conditions). In other words, the 5′ to 3′ nuclease activity of the polymerase is about one-fifth, or less than about one-fifth, the 5′ to 3′ nuclease activity of Taq. For example, the polymerase can have a 5′ to 3′ nuclease activity that is ten percent or less (e.g., five percent or less) than that of Taq under typical reaction conditions. Optionally, the polymerase has no detectable 5′ to 3′ nuclease activity under typical reaction conditions (e.g., typical PCR conditions).

In one class of embodiments, the hairpin primer is resistant to 5′ to 3′ nuclease activity. A variety of nuclease-resistant hairpin primers can be created, e.g., comprising modified nucleotides or modified internucleotide linkages such as those used in the synthesis of antisense oligonucleotides. For example, the hairpin primer can comprise a peptide nucleic acid (PNA). As another example, the hairpin primer can comprise one or more 2′-O-methyl nucleotides (e.g., a hairpin primer comprising standard deoxyribonucleotides can also comprise one or more 2′-O-methyl nucleotides, e.g., at its 5′ end, or a hairpin primer can consist entirely of 2′-O-methyl nucleotides). The hairpin primer can contain one or more modified internucleotide linkages, as well as or instead of one or more modified nucleotides. For example, the hairpin primer can comprise one or more phosphorothioate linkages (oligonucleotides comprising such linkages are sometimes called S-oligos). A hairpin primer can comprise, e.g., only phosphorothioate linkages or a mixture of phosphodiester and phosphorothioate linkages. As other examples, the hairpin primer can comprise one or more methylphosphonate linkages, one or more boranophosphate linkages, or the like. Combinations of typical nuclease resistance modification strategies can also be employed; for example, a nuclease resistant hairpin primer can comprise both 2′ -O-methyl nucleotides and phosphorothioate linkages. In other embodiments, the hairpin primer includes only standard nucleotides and phosphodiester bonds and is not resistant to 5′ to 3′ nuclease activity.

The template nucleic acid can be, e.g., any single-stranded or double-stranded DNA. For example, in one embodiment, the template nucleic acid is a single-stranded DNA product of a reverse transcription reaction (e.g., FHPs can be conveniently used to detect RNA targets by reverse transcription-PCR, including quantitative reverse transcription-PCR). As other examples, the template nucleic acid can be a synthetic oligonucleotide, a double-stranded cDNA, a single-stranded PCR product, or a double-stranded PCR product or can comprise genomic DNA.

The template nucleic acid can be derived from essentially any source, including but not limited to: a human; an animal; a plant; a bacterium; a virus; cultured cells or culture medium; a tissue or fluid, e.g., from a patient, such as skin, blood, sputum, urine, stool, semen, or spinal fluid; a tumor; a biopsy; and/or the like.

The composition can optionally also include other reagents required to amplify a nucleic acid target, for example, deoxyribonucleotides triphosphates, an aqueous buffer, appropriate salts and metal cations, and/or the like.

In one aspect, the invention includes systems and devices for use of the compositions, e.g., according to the methods herein. In one class of embodiments, the composition is contained in a thermal cycler (e.g., in one or more sample tubes or one or more wells of a multiwell plate, in a reaction region of a thermal cycler, e.g., an automated thermal cycler). The system can include, e.g., a computer with appropriate software for controlling the operation of the thermal cycler (e.g., temperature and duration of each step, ramping between steps, and/or number of cycles) coupled to the thermal cycler. Similarly, the system can include a detector coupled to the thermal cycler and/or computer (e.g., for measuring the fluorescence spectrum and/or intensity from one or more wells of a multiwell plate contained in the reaction region of the thermal cycler after excitation by laser light source).

The computer typically includes appropriate software for receiving user instructions, either in the form of user input into a set of parameter fields, e.g., in a GUI, or in the form of preprogrammed instructions, e.g., preprogrammed for a variety of different specific operations. The software optionally converts these instructions to appropriate language for instructing the operation of the thermal cycler to carry out the desired operation. The computer can also receive data from the thermal cycler and/or detector regarding fluorescent intensity, cycle completion or the like and can interpret the data, provide it to a user in a human readable format, or use that data to initiate further operations (e.g., additional thermal cycles), in accordance with any programming by the user.

Another aspect of the invention provides kits. Thus, one general class of embodiments provides a kit for use in amplifying a target nucleotide sequence or its reverse complement from a template nucleic acid strand that comprises a target region comprising the target nucleotide sequence or its reverse complement. The kit includes a first linear primer, a second linear primer, and a hairpin primer, packaged in one or more containers. The first linear primer comprises a region of identity to a 5′ subregion of the target region, while the second linear primer comprises a region of complementarity to a 3′ subregion of the target region. The 5′ subregion is located at the 5′ end of the target region and the 3′ subregion is located at the 3′ end of the target region; thus the first and second linear primers define the two ends of the target region. The hairpin primer comprises a region of complementarity to a first subregion of the target region, the first subregion being 5′ of or at least partially overlapping the 3′ subregion.

In one class of embodiments, the hairpin primer comprises a 5′ arm, a loop and a 3′ arm. The 5′ arm and the 3′ arm are complementary to each other and are able to form a double-stranded duplex. At least a portion of the loop and the 3′ arm are complementary to the first subregion of the target region. The entire 3′ arm is typically complementary to the first subregion; the entire loop is typically but not necessarily also complementary to the first subregion. The 5′ arm can be partially or entirely complementary to the target region as well, but typically is not. Thus, in a preferred class of embodiments, the entire loop and 3′ arm are complementary to the first subregion.

In one class of embodiments, the hairpin primer consists of the 5′ arm, loop and 3′ arm, with at least a portion of the loop and the 3′ arm being complementary to the first subregion; that is, the double-stranded duplex formed by the arms is blunt-ended with no overhanging 3′ or 5′ nucleotides. In other embodiments, additional sequence complementary to the target follows the 3′ arm.

The kit can include a fluorogenic hairpin primer. Thus, in one class of embodiments, the hairpin primer also includes a fluorescent label and a quencher. The label emits a fluorescent signal. The label and the quencher are located within the hairpin primer such that the label emits a maximal fluorescent signal only when the 5′ and 3′ arms are not forming the double-stranded duplex.

The first and second linear primers can be at equal concentrations. As noted above, however, providing an excess of the first linear primer can enhance incorporation of the hairpin primer (and thus increase sensitivity of target detection by a FHP) in an amplification reaction. Thus, in one class of embodiments, the first linear primer is provided a concentration that is at least about 1.3 times (e.g., at least about two times, at least about three times, at least about four times, at least about five times, at least about six times, or more) the concentration of the second linear primer.

In one class of embodiments, the primers are fully nested; that is, the first subregion does not overlap the 3′ subregion. In these embodiments, the primers do not compete for annealing to the template. In another class of embodiments, the first subregion at least partially overlaps the 3′ subregion. The second linear primer and the hairpin primer thus compete for annealing to the first strand of the template in these embodiments. The first and 3′ subregions can partially or completely overlap, although typically the 5′ end of the first subregion is 5′ of the 5′ end of the 3′ subregion and the 3′ end of the first subregion is 5′ of the 3′ end of the second subregion.

The hairpin primer is optionally resistant to 5′ to 3′ nuclease activity. A variety of nuclease-resistant hairpin primers can be created, e.g., as described above. In other embodiments, the hairpin primer includes only standard nucleotides and phosphodiester bonds and is not resistant to 5′ to 3′ nuclease activity.

The kit optionally also includes one or more of: a polymerase (e.g., a polymerase having or substantially lacking 5′ to 3′ nuclease activity), a buffer, a standard template for calibrating a detection reaction, instructions for extending the hairpin primer to amplify at least a portion of the target nucleotide sequence or reverse complement thereof, instructions for using the components to amplify, detect and/or quantitate the target nucleotide sequence or reverse complement thereof, or packaging materials.

Fluorescent probes form another feature of the invention. One class of embodiments provides a hairpin probe comprising or consisting of a 5′ arm, a loop, a 3′ arm, a fluorescent label that emits a fluorescent signal, and a quencher. The 5′ arm and the 3′ arm are complementary to each other and are able to form a double-stranded duplex. The double-stranded duplex formed by the 5′ and 3′ arms has a Tm that is equal to or less than a preselected detection temperature at which the fluorescent signal is detected (e.g., at least about 1° C., at least about 2° C., or at least about 5° C. less than the detection temperature, e.g., between about 5° C. and about 10° C. less than the detection temperature). At least a portion of the loop and the 3′ arm (preferably the entire loop and 3′ arm) are complementary to a portion of a target nucleotide sequence or its reverse complement. The label and the quencher are located within the probe such that the label emits a maximal fluorescent signal only when the 5′ and 3′ arms are not forming the double-stranded duplex. The probe is not extendable, and thus either the label or the quencher can optionally be located at the 3′ end of the probe, blocking the 3′ hydroxyl.

Molecular Biological Techniques

In practicing the present invention, many conventional techniques in molecular biology, microbiology, and recombinant DNA technology are optionally used. These techniques are well known and are explained in, for example, Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (3rd Ed.), Vol. 1-3, Cold Spring Harbor Laboratory, Cold Spring Harbor, N.Y., 2000 (“Sambroo”) and Current Protocols in Molecular Biology, F. M. Ausubel et al., eds., Current Protocols, a joint venture between Greene Publishing Associates, Inc. and John Wiley & Sons, Inc., (supplemented through 2002) (“Ausubel”)). Other useful references, e.g. for cell isolation and culture (e.g., for subsequent nucleic acid or protein isolation) include Freshney (1994) Culture of Animal Cells, a Manual of Basic Technique, third edition, Wiley-Liss, New York and the references cited therein; Payne et al. (1992) Plant Cell and Tissue Culture in Liquid Systems John Wiley & Sons, Inc. New York, N.Y.; Gamborg and Phillips (Eds.) (1995) Plant Cell, Tissue and Organ Culture; Fundamental Methods Springer Lab Manual, Springer-Verlag (Berlin Heidelberg New York) and Atlas and Parks (Eds.) The Handbook of Microbiological Media (1993) CRC Press, Boca Raton, Fla.

Nucleic Acid Amplification

Nucleic acid amplification by template-directed, enzyme-dependent extension of primers is well known in the art. For example, amplification by the polymerase chain reaction (PCR) has been described. Details regarding various PCR methods, including, e.g., asymmetric PCR, reverse transcription-PCR, in situ PCR, quantitative PCR, real time PCR, and multiplex PCR, are well described in the literature. Details regarding PCR methods and applications thereof are found, e.g., in Sambrook et al., Molecular Cloning—A Laboratory Manual (3rd Ed.), Vol. 1-3, Cold Spring Harbor Laboratory, Cold Spring Harbor, N.Y. (2000); F. M. Ausubel et al. (eds.), Current Protocols in Molecular Biology, Current Protocols, a joint venture between Greene Publishing Associates, Inc. and John Wiley & Sons, Inc., (supplemented through 2002); Innis et al. (eds.), PCR Protocols: A Guide to Methods and Applications, Academic Press Inc., San Diego, Calif. (1990); J. P. V. Heuvel, PCR Protocols in Molecular Toxicology, CRC Press (1997); H. G. and A. Griffin, PCR Technology: Current Innovations, CRC Press (1994); Bagasra et al., (1997) In Situ PCR Techniques, Jossey-Bass; Bustin (2000) “Absolute quantification of mRNA using real-time reverse transcription polymerase chain reaction assays” Journal of Molecular Endocrinology 25:169-193; Poddar (2000) “Symmetric vs. asymmetric PCR and molecular beacon probe in the detection of a target gene of adenovirus” Molecular and Cellular Probes 14: 25-32; and Mackay et al. (2002) “Real-time PCR in virology” Nucleic Acids Res. 30:1292-1305, and references therein, among many other references. Additional details regarding PCR methods, including asymmetric PCR methods, are found in the patent literature, e.g., U.S. Pat. No. 6,391,544 (May 21, 2002) to Salituro et al. entitled “Method for using unequal primer concentrations for generating nucleic acid amplification products”; U.S. Pat. No. 5,066,584 (Nov. 19, 1991) to Gyllensten et al. entitled “Methods for generating single stranded DNA by the polymerase chain reaction”; U.S. Pat. No. 5,691,146 (Nov. 25, 1997) to Mayrand entitled “Methods for combined PCR amplification and hybridization probing using doubly labeled fluorescent probes”; and U.S. patent application Ser. No. 10/281,054 (filed Oct. 24, 2002) by Beckman et al. entitled “Asymmetric PCR with nuclease-free polymerase or nuclease-resistant molecular beacons.”

In brief, PCR typically uses at least one pair of primers (typically synthetic oligonucleotides). Each primer hybridizes to a strand of a double-stranded nucleic acid target that is amplified (the original template may be either single-stranded or double-stranded). A pair of primers typically flanks a nucleic acid target that is amplified. In this invention where three primers are used, the first and second linear primers define one amplicon comprising the entire target region, and the first linear primer and the hairpin primer define another amplicon comprising at least a portion of the target region. Template-dependent extension of the primers is catalyzed by a DNA polymerase, in the presence of deoxyribonucleoside triphosphates (typically dATP, dCTP, dGTP, and dTTP, although these can be replaced and/or supplemented with other dNTPs, e.g., a dNTP comprising a base analog that Watson-Crick base pairs like one of the conventional bases, e.g., uracil, inosine, or 7-deazaguanine), an aqueous buffer, and appropriate salts and metal cations (e.g., Mg²⁺). The PCR process typically involves cycles of three steps: denaturation (e.g., of double-stranded template and/or extension product), annealing (e.g., of one or more primers to template), and extension (e.g., of one or more primers to form double-stranded extension products). The PCR process can instead, e.g., involve cycles of two steps: denaturation (e.g., of double-stranded template and/or extension product) and annealing/extension (e.g., of one or more primers to template and of one or more primers to form double-stranded extension products). The cycles are typically thermal cycles; for example, cycles of denaturation at temperatures greater than about 90° C., annealing at 50-75° C., and extension at 60-78° C. A thermostable enzyme is thus preferred. Automated thermal cyclers, including integrated systems for real time detection of product, are commercially available, e.g., the ABI Prism® 7700 sequence detection system from Applied Biosystems (www.appliedbiosystems.com), the iCycler iQ® real-time PCR detection system from Bio-Rad (www.biorad.com), or the DNA Engine Opticon® continuous fluorescence detection system from MJ Research, Inc. (www.mjr.com). Thermostable enzymes (including Thermus aquaticus Taq DNA polymerase, as well as enzymes substantially lacking 5′ to 3′ nuclease activity), appropriate buffers, etc. are also widely commercially available, e.g., from Clontech (www.clontech.com), Invitrogen (www.invitrogen.com), Sigma-Aldrich (www.sigma-aldrich.com), and New England Biolabs (www.neb.com). For example, thermostable polymerases lacking 5′ to 3′ nuclease activity are commercially available, e.g., Titanium® Taq (Clontech, www.clontech.com), KlenTaq DNA polymerase (Sigma-Aldrich, www.sigma-aldrich.com), Vent® and DeepVent® DNA polymerase (New England Biolabs, www.neb.com), and Tgo DNA polymerase (Roche, www.roche-applied-science.com).

A number of variations on the basic PCR technique are known in the art and can be adapted to the practice of this invention. For example, in in situ PCR, PCR amplification is performed in fixed cells, and the amplified target can remain largely within the cell (or organelle etc.) which originally contained the nucleic acid template. Quantitative PCR can be employed, e.g., to determine the amount (relative or absolute) of target initially present in a sample. In real time PCR, product formation is monitored in real time. In real time quantitative PCR with fluorescent detection of product, a fluorescence threshold above background is typically assigned, and the time point at which each reaction's amplification plot reaches that threshold (defined as the threshold cycle number or Ct) is determined. The Ct value can be used to calculate the quantity of template initially present in each reaction. (Under a standard set of conditions, a lower or higher starting template concentration produces a higher or lower, respectively, Ct value.) In multiplex PCR, multiple target sequences can be amplified, detected, and/or quantitated simultaneously in one reaction mixture. In reverse transcription-PCR, reverse transcription of an RNA (e.g., an mRNA) produces a single-stranded DNA template that is used in subsequent PCR cycles. Combinations of such techniques (e.g., quantitative real time reverse transcription-PCR) are routine.

Primer Design and Synthesis

Design of linear primers for nucleic acid amplification is routine for one of skill. Design of PCR primers, for example, is described in many of the above references that detail PCR methods.

Design of hairpin primers, including fluorogenic hairpin primers, has also been described, herein and in, e.g., U.S. Pat. No. 6,277,607 (Aug. 21, 2001) to Tyagi et al. entitled “High specificity primers, amplification methods and kits”; U.S. Pat. No. 5,866,336 (Feb. 2, 1999) to Nazarenko et al. entitled “Nucleic acid amplification oligonucleotides with molecular energy transfer labels and methods based thereon”; Kaboev et al. (2000) Nuc. Acids Res. 28 (21):e94; and Nazarenko et al. (1997) “A closed tube format for amplification and detection of DNA based on energy transfer” Nucl. Acids Res. 25:2516-2521.

Design of hairpin primers (e.g., FHPs) is preferably performed using software which enables themodynamic modeling of the hairpin structure, e.g., Mfold DNA server (http://www.bioinfo.rpi.edu/applications/mfold/old/dna/form1.ci). Preferably, the sequence of the HP is designed using such software such that there is only a single predicted stable secondary structure for the HP, i.e. only one predicted structure containing a self-complementary double-stranded region that has a negative free energy (ΔG), such that this one structure is the desired HP, e.g., with perfectly complementary 5′ and 3′ arms that form a blunt-ended hairpin structure. Stem lengths can be, e.g., between 4 and 15 nt (nucleotides) in length, preferably between 5 and 10 nt in length, and even more preferably between 6 and 9 nt in length. Depending upon the GC content of the stem, the Tm of the stem can be, e.g., between about 20° C. less than and about 30° C. greater than a preselected detection temperature at which the signal is detected (e.g., a PCR annealing temperature), preferably between 5° C. less than and 20° C. greater than the detection temperature, and more preferably between 5° C. greater than and 15° C. greater than the detection temperature (e.g., for a PCR annealing temperature of 55° C., the Tm of the stem can be between 35° C. and 85° C., preferably between 50° C. and 75° C., and more preferably between 60° C. and 70° C.).

Depending upon the GC content of the first subregion, the number of nucleotides of the hairpin primer that are complementary to the first strand of the template can be chosen such that the Tm of the hairpin primer (e.g., the loop and 3′ arm)-first subregion duplex is, e.g., between about 5° C. less than and about 25° C. greater than the annealing temperature, and preferably between about 5° C. greater than and about 20° C. greater than the annealing temperature (e.g., for a PCR annealing temperature of 55° C., the hairpin primer-first strand Tm can be between about 50° C. and about 80° C., preferably between about 60° C. and about 75° C.).

Increasing the length of the stem (e.g., from 5 nt to 9 nt) can increase specificity of priming by the hairpin primer; it can also decrease efficiency of priming by the hairpin primer, particularly in early PCR cycles when the concentration of template is low. Use of the first and second linear primers, however, results in efficient amplification of the template in these early cycles, and as the concentration of template increases, mass action can help drive annealing and incorporation of the hairpin primer into product. Use of the linear primers thus enables the use of a hairpin primer (e.g., a FHP) that might otherwise not be efficiently extended.

As noted, a fluorogenic hairpin primer includes a fluorescent label/quencher pair. Either the label or the quencher is attached to the 5′ arm while the other member of the pair is attached to the 3′ arm (leaving a free 3′ hydroxyl, such that the primer is extendable by a polymerase). As one example, a FAM fluorophore can be attached to the 5′ end of the primer and a DABCYL quencher can be attached at various positions in the 3′ arm (leaving the 3′ hydroxyl free).

FHPs can incorporate any of a variety of fluorophore/quencher combinations, using e.g., fluorescence resonance energy transfer (FRET)-based quenching, non-FRET based quenching, or wavelength-shifting harvester molecules. Example combinations include terbium chelate and TRITC (tetrarhodamine isothiocyanate), europium cryptate and Allophycocyanin, fluorescein and tetramethylrhodamine, IAEDANS and fluorescein, EDANS and DABCYL, fluorescein and DABCYL, fluorescein and fluorescein, BODIPY FL and BODIPY FL, and fluorescein and QSY 7 dye. Nonfluorescent acceptors such as DABCYL and QSY 7 and QSY 33 dyes have the particular advantage of eliminating background fluorescence resulting from direct (i.e., nonsensitized) acceptor excitation. A variety of probes incorporating fluorescent donor-nonfluoresent acceptor combinations have been developed for detection of nucleic acid hybridization events. See e.g., Haugland Handbook of Fluorescent Probes and Research Chemicals, Ninth Edition published by Molecular Probes, Inc., Eugene, Oreg.) or a more current on-line (www.probes.com) or CD-ROM version of the Handbook (available from Molecular Probes, Inc.). Detectable signals from such FHPs include changes in fluorescence and/or changes in absorption spectra.

Absorption by or fluorescent emissions from FHPs can be detected by essentially any method known in the art. In the context of real time PCR, for example, fluorescent emissions can be conveniently detected during the amplification by use of a commercially available integrated system such as, e.g., the ABI Prism® 7700 sequence detection system from Applied Biosystems (www.appliedbiosystems.com), the iCycler iQ® real-time PCR detection system from Bio-Rad (www.biorad.com), or the DNA Engine Opticon® continuous fluorescence detection system from MJ Research, Inc. (www.mjr.com).

FHPs can be synthesized using conventional methods. For example, oligos can be synthesized on commercially available automated oligonucleotide synthesis machines using standard methods. Labels can be attached to the oligos either during automated synthesis or by post-synthetic reactions which have been described before; see, e.g., Tyagi and Kramer (1996) “Molecular beacons: probes that fluoresce upon hybridization” Nature Biotechnology 14:303-308; Nelson, et al. (1989) “Bifunctional Oligonucleotide Probes Synthesized Using A Novel CPG Support Are Able To Detect Single Base Pair Mutations” Nucleic Acids Research 17:7187-7194; U.S. Pat. No. 6,277,607 (Aug. 21, 2001) to Tyagi et al. entitled “High specificity primers, amplification methods and kits”; U.S. Pat. No. 6,037,130 to Tyagi et al (Mar. 14, 2000), entitled “Wavelength-shifting probes and primers and their use in assays and kits”; and U.S. Pat. No. 5,925,517 (Jul. 20, 1999) to Tyagi et al. entitled “Detectably labeled dual conformation oligonucleotide probes, assays and kits.”

For example, a label or quencher can be incorporated during oligonucleotide synthesis by using a specialized phosphoramidite including the label or quencher, or a modified base phosphoramidite including an alkyl spacer can be incorporated during oligonucleotide synthesis and the label or quencher can be linked to the spacer after synthesis is complete. As a specific example, fluorescein can be incorporated at the 5′ end of a FHP by using a fluorescein phosphoramidite in the last step of the synthesis. As another specific example, a modified T including a C6 spacer with a primary amino group can be incorporated into the oligonucleotide, and a succinimidyl ester of 4-(4′-dimethylaminophenylazo)benzoic acid (DABCYL) can be attached to the primary amino group. (Such modified phosphoramidites are commercially available, e.g., AminoModifier C6 dT from Glen Research.) Similarly, 4-dimethylaminophenylazophenyl-4′-maleimide (DABMI) can be used when the site of attachment is a sulphydryl group. As other examples, fluorescein can be introduced into oligos, either by using a fluorescein phosphoramidite that replaces a nucleoside with fluorescein, or by using a fluorescein dT phosphoramidite that introduces a fluorescein moiety at a thymidine ring via a spacer. To link a fluorescein moiety to a terminal location, iodoacetoamidofluorescein can be coupled to a sulphydryl group. Tetrachlorofluorescein (TET) can be introduced during automated synthesis using a 5′-tetrachloro-fluorescein phosphoramidite. Other reactive fluorophore derivatives and their respective sites of attachment include the succinimidyl ester of 5-carboxyrhodamine-6G (RHD) coupled to an amino group; an iodoacetamide of tetramethylrhodamine coupled to a sulphydryl group; an isothiocyanate of tetramethylrhodamine coupled to an amino group; or a sulfonylchloride of Texas red coupled to a sulphydryl group.

Optimal placement of the label and quencher within a FHP (e.g., for maximal quenching of the label when the primer is in its hairpin conformation) can readily be determined by one of skill using techniques known in the art. For example, the label and quencher can be located at complementary nucleotide positions within the 5′ and 3′ arms, or the quencher can be five nucleotides away (approximately one-half helical turn) from the position of the label on the other arm. For example, for a FHP consisting of a loop and 5′ and 3′ arms, the fluorophore can be attached at the 5′ terminus and the quencher can be attached to the 3′ base. Quenching of the label can verified by checking the absorbance and/or fluorescence spectrum of the FHP at a temperature below the expected Tm of the duplex formed by the arms. Similarly, signal from the label can be verified at a temperature above the expected Tm of the arm duplex.

In general, synthetic methods for making oligonucleotides (including labeled oligos) are well known. For example, oligonucleotides can be synthesized chemically according to the solid phase phosphoramidite triester method described by Beaucage and Caruthers (1981), Tetrahedron Letts., 22(20):1859-1862, e.g., using a commercially available automated synthesizer, e.g., as described in Needham-VanDevanter et al. (1984) Nucleic Acids Res., 12:6159-6168. Synthesis of modified oligonucleotides (e.g., oligonucleotides comprising 2′-O-methyl nucleotides and/or phosphorothioate, methylphosphonate, or boranophosphate linkages, e.g., for use as nuclease resistant primers) are described in e.g., Oligonucleotides and Analogs (1991), IRL Press, New York; Shaw et al. (1993), Methods Mol. Biol. 20:225-243; Nielsen et al. (1991), Science 254:1497-1500; and Shaw et al. (2000) Methods Enzymol. 313:226-257.

Oligonucleotides, including modified oligonucleotides (e.g., oligonucleotides comprising fluorophores and quenchers, 2′-O-methyl nucleotides, and/or phosphorothioate, methylphosphonate, or boranophosphate linkages) can also be ordered from a variety of commercial sources known to persons of skill. There are many commercial providers of oligo synthesis services, and thus, this is a broadly accessible technology. Essentially any nucleic acid can be custom ordered from any of a variety of commercial sources, such as The Midland Certified Reagent Company (www.mcrc.com), The Great American Gene Company (www.genco.com), ExpressGen Inc. (www.expressgen.com), QIAGEN (http://oligos.qiagen.com) and many others.

After synthesis, a primer (e.g., a linear primer or a FHP) can be purified, if desired, e.g., by high pressure liquid chromatography or other methods known in the art.

While the foregoing invention has been described in some detail for purposes of clarity and understanding, it will be clear to one skilled in the art from a reading of this disclosure that various changes in form and detail can be made without departing from the true scope of the invention. For example, all the techniques and compositions described above can be used in various combinations. It is understood that the examples and embodiments described herein are for illustrative purposes only and that various modifications or changes in light thereof will be suggested to persons skilled in the art and are to be included within the spirit and purview of this application and scope of the appended claims.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. A method for amplifying a target nucleotide sequence or reverse complement thereof, the method comprising:
providing a template nucleic acid comprising a first strand, the first strand comprising a target region that comprises the target nucleotide sequence or its reverse complement;
providing a first linear primer comprising a region of identity to a 5′ subregion of the target region;
providing a second linear primer comprising a region of complementarity to a 3′ subregion of the target region;
providing a hairpin primer comprising a region of complementarity to a first subregion of the target region, the first subregion being 5′ of or at least partially overlapping the 3′ subregion;
contacting the template nucleic acid, the first linear primer, the second linear primer and the hairpin primer; and,
extending at least the hairpin primer, thereby amplifying at least a portion of the target nucleotide sequence or its reverse complement.

2. The method of claim 1, wherein the hairpin primer comprises a 5′ arm, a loop and a 3′ arm, the 5′ arm and the 3′ arm being complementary to each other and able to form a double-stranded duplex, and at least a portion of the loop and the 3′ arm being complementary to the first subregion of the target region.

3. The method of claim 2, wherein the hairpin primer consists of a 5′ arm, a loop and a 3′ arm, the 5′ arm and the 3′ arm being complementary to each other and able to form a double-stranded duplex, and at least a portion of the loop and the 3′ arm being complementary to the first subregion of the target region.

4. The method of claim 2, wherein the entire loop and 3′ arm are complementary to the first subregion.

5. The method of claim 2, wherein the hairpin primer comprises a fluorescent label that emits a fluorescent signal and a quencher, the label and the quencher being located within the hairpin primer such that the label emits a maximal fluorescent signal only when the 5′ and 3′ arms are not forming the double-stranded duplex.

6. The method of claim 5, wherein incorporation of the hairpin primer into a double-stranded product of the extending step prevents formation of the double-stranded duplex and thus permits emission of the maximal fluorescent signal.

7. The method of claim 5, comprising detecting the fluorescent signal emitted by the label.

8. The method of claim 7, wherein detecting the fluorescent signal comprises measuring the intensity of the fluorescent signal.

9. The method of claim 5, wherein:
providing a first linear primer comprises providing two or more first linear primers, each of which comprises a region of identity to a 5′ subregion of a different target region;
providing a second linear primer comprises providing two or more second linear primers, each of which comprises a region of complementarity to a 3′ subregion of a different target region;
providing a hairpin primer comprises providing two or more hairpin primers, each of which comprises a region of complementarity to a first subregion of a different target region, the first subregion being 5′ of or at least partially overlapping the 3′ subregion of that target region;
providing a template nucleic acid comprises providing one or more template nucleic acids collectively comprising the different target regions; and,
extending at least the hairpin primer comprises extending at least each of the hairpin primers.

10. The method of claim 9, wherein each hairpin primer comprises a different fluorescent label that emits a fluorescent signal distinguishable from that of each of the other fluorescent labels, the method comprising detecting the fluorescent signal from the label on each of the two or more hairpin primers.

11. The method of claim 1, comprising extending the first and second linear primers.

12. The method of claim 1, wherein the first linear primer is provided a concentration that is at least about 1.3 times that of the second linear primer.

13. The method of claim 12, wherein the first linear primer is provided a concentration that is at least about two times that of the second linear primer.

14. The method of claim 12, wherein the first linear primer is provided a concentration that is at least about three times that of the second linear primer.

15. The method of claim 1, wherein the first subregion at least partially overlaps the 3′ subregion.

16. The method of claim 1, wherein the first subregion does not overlap the 3′ subregion.

17. The method of claim 1, wherein extending at least the hairpin primer comprises using a polymerase substantially lacking 5′ to 3′ nuclease activity to extend at least the hairpin primer.

18. The method of claim 1, wherein the hairpin primer is resistant to 5′ to 3′ nuclease activity.

19. The method of claim 1, wherein the template nucleic acid is a single-stranded DNA product of a reverse transcription reaction.

20. The method of claim 1, wherein the template nucleic acid is a double-stranded cDNA, a single-stranded PCR product, or a double-stranded PCR product.

21. The method of claim 1, wherein the template nucleic acid comprises genomic DNA.

22. A composition comprising:
a template nucleic acid comprising a first strand, the first strand comprising a target region that comprises a target nucleotide sequence or its reverse complement;
a first linear primer comprising a region of identity to a 5′ subregion of the target region;
a second linear primer comprising a region of complementarity to a 3′ subregion of the target region; and,
a hairpin primer comprising a region of complementarity to a first subregion of the target region, the first subregion being 5′ of or at least partially overlapping the 3′ subregion.

23. The composition of claim 22, wherein the hairpin primer comprises a 5′ arm, a loop and a 3′ arm, the 5′ arm and the 3′ arm being complementary to each other and able to form a double-stranded duplex, and at least a portion of the loop and the 3′ arm being complementary to the first subregion of the target region.

24. The composition of claim 23, wherein the hairpin primer consists of a 5′ arm, a loop and a 3′ arm, the 5′ arm and the 3′ arm being complementary to each other and able to form a double-stranded duplex, and at least a portion of the loop and the 3′ arm being complementary to the first subregion of the target region.

25. The composition of claim 23, wherein the entire loop and 3′ arm are complementary to the first subregion.

26. The composition of claim 23, wherein the hairpin primer comprises a fluorescent label that emits a fluorescent signal and a quencher, the label and the quencher being located within the hairpin primer such that the label emits a maximal fluorescent signal only when the 5′ and 3′ arms are not forming the double-stranded duplex.

27. The composition of claim 26, wherein the hairpin primer is extended to form a double-stranded product, such incorporation of the hairpin primer into the product preventing formation of the double-stranded duplex and thus permitting emission of the maximal fluorescent signal.

28. The composition of claim 22, wherein the first linear primer is provided a concentration that is at least about 1.3 times that of the second linear primer.

29. The composition of claim 28, wherein the first linear primer is provided a concentration that is at least about two times that of the second linear primer.

30. The composition of claim 28, wherein the first linear primer is provided a concentration that is at least about three times that of the second linear primer.

31. The composition of claim 22, wherein the first subregion at least partially overlaps the 3′ subregion.

32. The composition of claim 22, wherein the first subregion does not overlap the 3′ subregion.

33. The composition of claim 22, comprising a polymerase.

34. The composition of claim 33, wherein the polymerase substantially lacks 5′ to 3′ nuclease activity.

35. The composition of claim 22, wherein the hairpin primer is resistant to 5′ to 3′ nuclease activity.

36. The composition of claim 22, wherein the template nucleic acid is a single-stranded DNA product of a reverse transcription reaction.

37. The composition of claim 22, wherein the template nucleic acid is a double-stranded cDNA, a single-stranded PCR product, or a double-stranded PCR product.

38. The composition of claim 22, wherein the template nucleic acid comprises genomic DNA.

39. The composition of claim 22, wherein the composition is contained in a thermal cycler.

40. A kit for use in amplifying a target nucleotide sequence or reverse complement thereof from a template nucleic acid strand comprising a target region that comprises the target nucleotide sequence or its reverse complement, the kit comprising:
a first linear primer comprising a region of identity to a 5′ subregion of the target region;
a second linear primer comprising a region of complementarity to a 3′ subregion of the target region; and,
a hairpin primer comprising a region of complementarity to a first subregion of the target region, the first subregion being 5′ of or at least partially overlapping the 3′ subregion,
packaged in one or more containers.

41. The kit of claim 40, wherein the hairpin primer comprises a 5′ arm, a loop and a 3′ arm, the 5′ arm and the 3′ arm being complementary to each other and able to form a double-stranded duplex, and at least a portion of the loop and the 3′ arm being complementary to the first subregion of the target region.

42. The kit of claim 41, wherein the hairpin primer consists of a 5′ arm, a loop and a 3′ arm, the 5′ arm and the 3′ arm being complementary to each other and able to form a double-stranded duplex, and at least a portion of the loop and the 3′ arm being complementary to the first subregion of the target region.

43. The kit of claim 41, wherein the entire loop and 3′ arm are complementary to the first subregion.

44. The kit of claim 41, wherein the hairpin primer comprises a fluorescent label that emits a fluorescent signal and a quencher, the label and the quencher being located within the hairpin primer such that the label emits a maximal fluorescent signal only when the 5′ and 3′ arms are not forming the double-stranded duplex.

45. The kit of claim 40, wherein the first subregion at least partially overlaps the 3′ subregion.

46. The kit of claim 40, wherein the first subregion does not overlap the 3′ subregion.

47. The kit of claim 40, wherein the hairpin primer is resistant to 5′ to 3′ nuclease activity.

48. The kit of claim 40, comprising one or more of: a polymerase, a buffer, a standard template for calibrating a detection reaction, instructions for extending the hairpin primer to amplify at least a portion of the target nucleotide sequence or reverse complement thereof, instructions for using the components to amplify, detect and/or quantitate the target nucleotide sequence or reverse complement thereof, or packaging materials.
